(19) United States
(12) Patent Application Publication
(10) Pub. No.: US 2012/0213845 A1
(43) Pub. Date: Aug. 23, 2012

(54) SKIN COMPOSITION COMPRISING AN EXFOLIATE, A BRIGHTENER AND A COLLAGEN BOOSTER, AND METHOD OF USING THE SAME

(76) Inventor: Eric F. Bernstein, Gladwyne, PA (US)

(21) Appl. No.: 13/276,696

(22) Filed: Oct. 19, 2011

Related U.S. Application Data
(60) Provisional application No. 61/394,621, filed on Oct. 19, 2010.

(51) Int. Cl. A61K 9/127; A61K 8/37; A61K 36/889; A61K 38/00; A61Q 17/04; A61K 31/401; A61K 31/366; A61K 31/19; A61Q 17/00; A61K 8/18; A61K 31/728

(52) U.S. Cl. 424/450; 424/59; 424/60; 424/725; 424/757; 514/18.8; 514/54; 514/423; 514/460; 514/574

(57) ABSTRACT

There is disclosed a composition that provides improved anti-aging benefits and creates a healthier skin using the combination of skin exfoliation, brightening, and collagen synthesis. In one embodiment, the composition comprises, a skin exfoliation ingredient such as citric acid and gluconolactone, at least one skin brightening ingredient, such licorice extract, and at least one collagen enhancing ingredient, such as hydroxyproline, bioactive polypeptides or combinations thereof. Depending on the end-use, various optional ingredients, such as moisturizers, anti-inflammatory agents, UVA/UVB protectants, and cleansers, may be added to the composition, which may take various forms, such as a cream, mousse, serum, or gel.

### This application claims benefit of U.S. Provisional Application No. 61/394,621, filed Oct. 19, 2010, the contents of which are incorporated herein by reference.

The present disclosure is related to skin care compositions that provide a combination of skin exfoliation, brightening, and collagen synthesis. The disclosed skin care compositions rely on citric acid, and at least two types of ingredients, including a) whitening/brightening ingredients and b) collagen boosting ingredients, to give superior anti-aging properties.

The composition may be provided in any cosmetically acceptable form, such as serums, creams, lotions, gels and mousses. In one embodiment, the serum may be in the form of a night-time serum, a repair and protect serum, a calm and restore serum, a day time serum, or a cleanser.

It has been found that skin exfoliation processes that are currently widely used in anti-aging products are not sufficient to provide superior anti-aging benefits. In particular, there is a need for a composition to improve anti-aging benefits. Therefore, there is disclosed a composition and method of using it to create a younger and healthier skin using triple effects—skin exfoliation, brightening, and collagen synthesis.

There is disclosed a skin care composition to give a superior anti-aging properties using citric acid in conjunction with at least two types of ingredients, including (a) whitening/brightening ingredients and (b) collagen boosting ingredients.

In addition to the foregoing ingredients, this basic composition may further comprise other beneficial components, which has been found to enhance anti-aging benefits, such as collagen promoters, super-moisturizers, skin brighteners, and anti-inflammatory agents. For example, in one embodiment, there is disclosed a citric acid based composition, as described above, further comprising:
a collagen promoter, such as hyaluronic acid (sodium hyaluronate);
a super-moisturizer agent, such as astrocaryum murumuru butter;
an anti-inflammatory agent, such as resveratrol; and
a fine line and wrinkle reducer, such as a peptide.

In one embodiment, the compositions described herein may be formed into a sunscreen composition. Thus, such a composition further comprises a sunscreen agent to provide a desired sun protection factor, such as an SPF of at least 15 or an SPF of at least 30.

There is also disclosed a skin cleanser composition comprising, in addition to the basic composition described above,
a skin exfoliation ingredient comprising citric acid and gluconolactone, and
a surfactant, such as a sulfate-free surfactant.

There is also disclosed a method of treating the skin that comprises administering to the skin an effective amount of a composition described above.

### DETAILED DESCRIPTION OF THE INVENTION

### Definitions

The compositions described herein are based on citric acid, which is a weak organic acid. More particularly, citric acid is an alpha-hydroxy acid having the formula (C₆H₈O₇). The 6 carbons associated with citric acid makes it a relatively large molecule, when compared to other cosmetic ingredients. Because of its large size, one would not expect it to absorb into the skin as effectively as it does. In addition, in biochemistry, the conjugate base of citric acid, citrate, is important as an intermediate in the citric acid cycle, and therefore occurs in the metabolism of virtually all living things.

Hyaluronic acid (sodium hyaluronate) is a naturally occurring substance that is a basic component of skin and some connective tissues. Hyaluronic acid promotes the formation of collagen and elastin. The presence of hyaluronic acid in epithelial tissue has been shown to promote keratinocyte proliferation, effecting skin hydration. As a result, hyaluronic acid increases the thickness of the epidermis causing the skin to look plump.

Licorice Root Extract (glycyrrhiza glabra) contains a variety of components that provide various cosmetic benefits to the skin, including glabridin which is skin whitening agent that helps decrease melanin in the epidermis.

Collagen boost ingredients used herein may comprise a bioactive amino acids and/or peptides (smaller than proteins). In one embodiment, the bioactive amino acid includes hydroxyproline (0.5%), which is a major component of the protein collagen and pre-collagen. Hydroxyproline plays a key role for collagen stability of pre-collagen and is cleaved in the process of making mature collagen. These cleavage products act as stimuli to cells, under normal conditions are indicative of dermal remodeling, thus stimulating dermal remodeling. In the case of a topical product, hydroxyproline will stimulate a healing response in the absence of a wound. Other collagen boosting ingredients used herein include milk complex proteins, such as whey protein and palmitoyl oligopeptide and palmitoyl tetrapeptide-7.

Anti-inflammatory agents used herein may comprise resveratrol (3,5,4′-trihydroxy-trans-stilbene), which is a stilbenoid, a type of natural phenol, and a phytoalexin produced naturally by several plants.

An agent that aids in diminishing the appearance of fine lines and wrinkles that may be used herein include peptides, which includes Palmitoyl Oligopeptide/Palmitoyl Tetrapeptide-7. Palmitoyl Oligopeptides is an amino-peptide, which comprise five amino acids linked together and attached to a fatty acid. Palmitoyl Oligopeptide is used herein to reduce wrinkle depth by increasing hyaluronic acid and collagen production.

In addition, light radiation with wavelengths between 280 and 320 nm, which are known as UVB rays, are known to cause skin burns and erythema. Rays with wavelengths of between 320 and 400 nm, which are known as UVA rays, induce damage to the skin, particularly leading to a loss of elasticity and the appearance of wrinkles, leading to premature ageing.

To avoid these adverse effects, cosmetic compositions for protecting the skin typically contain UVA and UVB screening agents. Therefore, in one embodiment, the compositions described herein may further contain a sunscreen agent.

As one skilled in the art would appreciate, screening agents (and the amounts thereof) are selected as a function of the desired sun protection factor (SPF), which is expressed mathematically as the ratio of the dose of UV radiation necessary to reach the erythema-forming threshold with the UV-screening agent to the dose of UV radiation necessary to reach the erythema-forming threshold without UV-screening agent.

In addition to creams, lotions, gels and mousses, the disclosed compositions may be in the form of a serum. As used herein, the term “serum” is understood to mean a cosmetic serum that is a highly concentrated product based on a water and oil emulsion, like other creams. However, serums contain much more active substances than creams. The cosmetic serums described herein act in the same way as creams in that they provide the above mentioned combination of properties. In contrast to creams, however, serum active substances assimilate easier and thus give the visible results quicker.

There is disclosed a skin care composition comprising, in a physiologically acceptable medium:
at least one skin exfoliation ingredient comprising citric acid;
at least one skin brightening ingredient, such as a licorice extract; and
at least one collagen enhancing ingredient, such as hydroxyproline, bioactive polypeptide or combinations thereof.

In one embodiment, the bioactive polypeptide comprises milk complex proteins, such as whey protein.

In one embodiment, the at least one skin exfoliation ingredient further comprises a polyhydroxy acid. When in the form of a cleanser, the skin exfoliation ingredient may also comprise gluconolactone.

It is appreciated that the compositions described herein may further comprise at least one moisturizing ingredient, such as hyaluronic acid, or derivatives thereof.

In one embodiment, the compositions described herein may further comprising at least one anti-inflammatory ingredient, such as resveratrol, butterbur extract, and combinations thereof.

In another embodiment, the composition may comprise at least one, and in some cases, all of the following components:
Citric Acid, such as in an amount ranging from 5-15% by weight, particularly 10% by weight;
Sodium Hyaluronate, such as in an amount ranging from 0.01 to 0.2% by weight; particularly 0.05% by weight;
Astrocaryum Murumuru Butter, such as in an amount ranging from 0.1 to 1.0% by weight, particularly 0.2% by weight;
Resveratrol, such as in an amount ranging from 0.1 to 2.0% by weight, particularly 1.0%;
Hydroxyproline, such as 0.1 to 1.0% by weight, particularly 0.5% by weight;
Whey Protein, such as 0.1 to 1.0% by weight, particularly 0.5% by weight;
Glycyrrhiza Glabra (Licorice) Root Extract, such as in an amount ranging from 0.01 to 0.2% by weight; particularly 0.05% by weight; and
Palmitoyl Oligopeptide/Palmitoyl Tetrapeptide-7, such as in an amount ranging from 0.5 to 5.0% by weight; particularly 3.0% by weight.

In one embodiment, the compositions described herein may be encapsulated in a traditional lecithin containing liposome, or in a unique delivery system. For example, the combination of a lecithin containing liposome and a red dye, allows the disclosed composition, when encapsulated therein, to bring the active ingredients to the skin deeper and faster than traditional delivery systems

There is also disclosed a skin care composition that is a cleanser, which comprises, in a physiologically acceptable medium:
a skin exfoliation ingredient comprising citric acid, optionally comprising gluconolactone;
at least one skin brightening ingredient, such as licorice extract;
at least one collagen enhancing ingredient, such as hydroxyproline, bioactive polypeptides or combinations thereof, and
a surfactant, such as a sulfate-free surfactant.

The sulfate-free surfactant may comprise coco glucoside and cocoyl sarcosine.

The cleanser embodiment may additionally comprise any one (or all) of the previously listed ingredients, such as at least one moisturizing ingredient, at least one anti-inflammatory ingredient, at least one antioxidant, hydrophilic/hydrophobic ingredient.

There is also disclosed a method of treating the skin, which comprises administering to the skin an effective amount of any of the compositions mentioned above to the skin. In one embodiment, the disclosed method comprises applying to the skin:
at least one skin exfoliation ingredient comprising citric acid;
at least one skin brightening ingredient; and
at least one collagen enhancing ingredient.

When made into a facial cleanser, the skin exfoliation ingredient comprises citric acid and gluconolactone, and the method comprises applying an effective amount of the composition to the skin, and removing it with water after cleansing.

When used as a sunscreen, the composition further comprises at least one ingredient that provides protection UVA/UVB protection to achieve an SPF factor of at least 15, such as 30 or greater. For example, in one embodiment, a sunscreen composition having an SPF of 30 comprises the following active ingredients:
Active Sunscreen Ingredients:
   Homosalate 10.00%
   Ethylhexyl Methoxycinnamate 7.60%
   Ethylhexyl Salicylate 5.00%
   Butyl Methoxydibenzoylmethane
   3.00%
   Octocrylene 2.79%
Cosmetically Active Ingredients:
   Citric Acid
   Palmitoyl Oligopeptide
   Palmitoyl Tetrapeptide-7
   Whey Protein
   Resveratrol
   Glycyrrhiza Glabra (Licorice) Root Extra
   Sodium Hyaluronate
   Astrocaryum Murumuru Butter
   Hydroxyproline
   Caffeine

As stated, compositions described herein may further comprise caffeine, which is a heterocyclic compound which is gaining popularity in cosmetics because of it has a rejuvenating effect on the skin. Caffeine has been found to firm and tighten the skin. Because it is a natural diuretic, it flushes fluids in the skin, thereby helping to tighten the skin, reduce puffiness (around the eyes, for example). It is also a known vasoconstrictor. Therefore, when applied to the skin, blood flow to that area is reduced thereby helping to reduce redness, such as a result of a sun burn. Caffeine also blocks UV radiation and contributes as a sunscreen.

A method of using the sunscreen composition comprises applying the which is in the form of a cream, mousse, serum, or gel to the skin.

### Additional Cosmetic Additives

The compositions of the present disclosure may include at least one cosmetic active principle, such as antioxidants, preservatives, fragrances, neutralizing agents, emollients, moisturizing agents, vitamins, and hydrophilic/hydrophobic ingredient such as green tea extract, green tea seed extract or combinations thereof.

It is to be understood that a person skilled in the art will take care to choose the at least one optional additional additive and/or the amounts thereof such that the advantageous properties of the composition according to the present disclosure are not, or are not substantially, detrimentally affected by the envisaged addition.

The composition according to the present disclosure may be packaged in a container comprising at least one compartment which comprises a composition of the present disclosure, the container being closed by a closure part. The container may be equipped with a means for the dispensing of the product, for example, the container can be equipped with a pump. In an alternative form, it can be equipped with a valve, the product then being packaged under pressure inside the container. In the cases where the container is equipped with a dispensing means, the presentation of the product in the foam form may be obtained at the time of the dispensing thereof.

The container may be at least partly made of a thermoplastic, for example, polypropylene and polyethylene. In an alternative embodiment, the container may be made of a nonthermoplastic, such as glasses, metals, and metal alloys.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements.

By way of non-limiting illustration, concrete examples of certain embodiments of the present disclosure are given below. Unless otherwise indicated, the amounts are given as percentage by weight.

### EXAMPLE

A clinical efficacy study was conducted with 31 female subjects to determine if the PM Focused Repair 4% described below used once nightly for 8 weeks helped to improve crow's feet fine lines/wrinkles, skin clarity/blotchiness, skin tone/evenness, pores and skin firmness/elasticity. The tested composition comprised the following active ingredients (in weight percent), in an physiologically/cosmetically acceptable medium:
Citric Acid—9.6%
Citric Acid 20% in Qusomes (water, citric acid and PEG 12 Glyceryl DiMyistate)—2.0
Sodium Hyaluronate—0.05%
Astrocaryum Murumuru Butter—0.20%
Resveratrol (gel of glycerin, resveratrol and lecithin)—1.0%
Hydroxyproline—0.50%
Whey Protein—0.50%
Glycyrrhiza Glabra (Licorice) Root Extract—0.050%
Palmitoyl Oligopeptide/Palmitoyl Tetrapeptide-7 (Matrixyl 3000™)—3.0%.

An aqueous solution of the Citric acid and Sodium Hyaluronate was mixed with 8.75% of a caustic soda 30% solution (water and sodium hydroxide) until a uniform mixture was achieved. The aqueous solution of citric acid further contained caffeine (0.20%) and glutathione (0.001%). At this time, the temperature of the batch was gradually increased to about 75° C.

Once this temperature was achieved, a previously mixed compound of xanthan gum (0.50%) and glycerin (5.0%) was added to the citric acid batch until uniformly dispersed therein.

Next, the Astrocaryum Murumuru Butter was added and mixed for about 15 minutes. After 15 minutes, the batch was slowly cooled to 65° C., and a phenoxyethanol (0.50%) was added and mixed until a uniform mixture occurred. At this time, cooling continued until 50° C.

When this temperature was reached, the Glycyrrhiza Glabra (Licorice) Root Extract was added and mixed until uniform. Once uniform, the batch was further cooled to 40° C., and the combination of Resveratrol and Citric Acid 20% in Qusomes was added and mixed until uniform.

In separate containers, aqueous solutions of Hydroxyproline and Whey Protein were premixed, and added to the main citric acid containing batch in sequential steps with the Hydroxyproline premix being added first, followed by the Whey Protein premix. After these ingredients were uniformly added to the batch, the Palmitoyl Oligopeptide/Palmitoyl Tetrapeptide-7 compound (Matrixyl 3000™) was added and mixing continued until smooth. Finally, 1.0% of an isoparaffin compound (C₁₃₋₁₄ Isoparaffin, polyacrylamide and Laureth-7) was added until the batch achieved a smooth and uniform consistency. Once achieved, the batch was cooled to room temperature.

The resulting product exhibited an initial viscosity of 13,000-18,000 cps and a 24 hour viscosity of 28,000-40,000 cps (as measured on a Brookfield RVT, measured at 5 RPM for 1 minute).

### A. Skin Evaluation Procedures

Evaluations of efficacy of this composition were based on a comparison of baseline vs. each observation period. Baseline Evaluations were performed in the following manner. Baseline tests were performed on a freshly washed “clean face” (without wearing face/eye area cosmetics or having applied any facial skin care products) for baseline visual assessments, instrumental measurements and digital photographs.

### 1. Skin Clarity/Blotchiness

Skin clarity was analyzed using the cross-polarized lighting of the Visia CR® system for image capture and the L*a*b* lighting analysis module from Canfield Scientific. The L* value, measuring black (0) to white (100) was used to indicate a change in skin clarity. An increase in the L* value indicated an improvement.

When images taken after 2, 4 and 8 weeks of product use were compared with baseline images, there was: a mean percent improvement of 0.3% in skin clarity/blotchiness after 2 weeks of product use, based on image analysis; a mean percent improvement of 1.0% in skin clarity/blotchiness after 4 weeks of product use, based on image analysis; and a mean percent worsening of 3.3% in skin clarity/blotchiness after 8 weeks of product use, based on image analysis. The changes observed after 4 and 8 weeks of product use were highly significant when compared with baseline.

### 2. Skin Tone/Evenness

In order to determine any changes in skin tone/color evenness, the CIE values (R=mean intensity of the red channel, G=mean intensity of the green channel and B=mean intensity of the blue channel) were analyzed. Value or brightness of a color is based on the amount of light emanating from a color. An increase in the value score indicated an improvement, an decrease indicated a worsening.

When images taken after 2, 4 and 8 weeks of product use were compared with baseline images, there was: a mean percent improvement of 1.2% in skin tone/evenness clarity after 2 weeks of product use, based on image analysis; a mean percent improvement of 1.7% in tone/evenness clarity after 4 weeks of product use, based on image analysis; and a mean percent worsening of 9.4% in skin tone/evenness clarity after 8 weeks of product use, based on image analysis. The changes observed after 2, 4 and 8 weeks of product use were statistically significant when compared with baseline.

### 3. Crow's Feet Fine Lines/Wrinkles

In order to determine changes in crow's feet fine lines/wrinkles, skin texture was analyzed. In order to analyze skin texture, each digital image was scanned horizontally and vertically to collect the red, green and blue intensities of the pixels. The proprietary mathematical algorithm in Visia CR® uses the pixel intensities of the scanned areas to calculate the texture score based on the totals of the mean intensities of the red, green and blue pixels. Texture scores are a single number calculated based on skin features. A decrease in the texture score represented an improvement in the appearance of crow's feet fine lines and wrinkles.

When images taken after 2, 4 and 8 weeks of product use were compared with baseline images, there was: a mean percent improvement of 0.1% in crow's feet fine lines/wrinkles after 2 weeks of product use, based on image analysis; a mean percent improvement of 7.2% in crow's feet fine lines/wrinkles after 4 weeks of product use, based on image analysis; and a mean percent worsening of 12.4% in crow's feet fine lines/wrinkles after 8 weeks of product use, based on image analysis. The changes observed after 4 and 8 weeks were statistically significant when compared with baseline.

### 4. Pore Size

In order to determine any changes in pore size, the mean pore area was analyzed (in pixels). A decrease in the mean area indicated an improvement, an increase indicated a worsening.

A trained technician took digital images on the face of each subject. Using ImagePro® Software, the images were analyzed to determine changes in pore size. When images taken after 2, 4 and 8 weeks of product use were compared with baseline images, there was: a mean percent improvement of 2.2% in pore size after 2 weeks of product use, based on image analysis; a mean percent improvement of 4.8% in pore size after 4 weeks of product use, based on image analysis; and a mean percent improvement of 1.6% in pore size after 8 weeks of product use, based on image analysis. The improvements observed after 2 and 4 weeks were highly significant when compared with baseline.

As shown, skin firmness was significantly improved after 2, 4 and 8 weeks of product use. In addition, skin clarity/blotchiness was significantly improved after 4 weeks of product use. Likewise, skin tone/evenness was significantly improved after 2 and 4 weeks of product use. Also, Crow's Feet Fine Line/Wrinkles were significantly improved after 4 weeks of product use. Finally, Pore Size was significantly improved after 2 and 4 weeks of product use.

There was no irritation observed during the course of the study. Rather, the product had a moderately high rate of consumer acceptance after 8 weeks of use. The majority of subjects reported an improvement in the appearance of crow's feet fine lines/wrinkles (81%), skin clarity (77%), skin imperfections/blotchiness (74%), skin tone (71%) and in the appearance of pores (77%). Additionally, 84% of subjects felt their skin was firmer and healthier since applying the product.

### 5. Skin Firmness-—Cutometer® Measurements

At each visit, a trained technician measured skin firmness on the face of each subject using the Cutometer®. An increase in Cutometer® measurements indicated an improvement (increase) in skin elasticity/resiliency. A decrease represented a worsening.

When measurements taken after 2, 4 and 8 weeks of product use were compared with baseline, there was: a mean percent improvement of 6.0% in skin firmness after 2 weeks of product use; a mean percent improvement of 10.0% in skin firmness after 4 weeks of product use; and a mean percent improvement of 13.0% in skin firmness after 8 weeks of product use. The improvements observed after 2, 4 and 8 weeks of product use were highly significant when compared with baseline. When compared with baseline, 100% of the subjects showed improvement in skin elasticity/firmness after 2 and 8 weeks of product use and 97% of the subjects showed improvement after 4 weeks.

What is claimed is:

1. A skin care composition comprising, in a physiologically acceptable medium:
at least one skin exfoliation ingredient comprising citric acid;
at least one skin brightening ingredient; and
at least one collagen enhancing ingredient.

2. The composition of claim 1, wherein said at least one skin brightening ingredient comprises a licorice extract.

3. The composition of claim 1, wherein said at least one collagen enhancing ingredient comprises hydroxyproline, bioactive polypeptide or combinations thereof.

4. The composition of claim 1, wherein said bioactive polypeptide comprises whey protein.

5. The composition of claim 1, wherein said at least one skin exfoliation ingredient further comprises a polyhydroxy acid.

6. The composition of claim 5, wherein said polyhydroxy acid comprises gluconolactone.

7. The composition of claim 1, further comprising at least one moisturizing ingredient chosen from hyaluronic acid, astrocaryum murumuru butter, and combinations thereof.

8. The composition of claim 1, further comprising at least one anti-inflammatory ingredient comprising resveratrol.

9. The composition of claim 1, wherein said composition is encapsulated in a lecithin containing liposome.

10. The composition of claim 1, further comprising at least one ingredient that provides protection UVA/UVB protection to achieve an SPF factor of at least 15.

11. The composition of claim 1, wherein said at least one ingredient that provides protection UVA/UVB protection comprises one or more of the following components:
Homosalate;
Ethylhexyl Methoxycinnamate;
Ethylhexyl Salicylate;
Butyl Methoxydibenzoylmethane; and
Octocrylene.

12. The composition of claim 1, further comprising at least one fine line and wrinkle reducer.

13. The composition of claim 1, wherein said at least one fine line and wrinkle reducer comprises a peptide chosen from Palmitoyl Oligopeptide, Palmitoyl Tetrapeptide-7, and combinations thereof.

14. The composition of claim 1, wherein said composition is in the form of a cream, lotion, mousse, serum, or gel.

15. A composition comprising:
at least one skin exfoliation ingredient comprising citric acid;
at least one skin brightening ingredient;
at least one collagen enhancing ingredient;
at least one collagen promoter;
at least one moisturizing agent;
at least one anti-inflammatory agent; and
at least one fine line and wrinkle reducer.

16. The composition of claim 15, wherein said at least one skin brightening ingredient comprises a licorice extract.

17. The composition of claim 15, wherein said at least one collagen enhancing ingredient comprises hydroxyproline, bioactive polypeptide or combinations thereof.

18. The composition of claim 17, wherein said bioactive polypeptide comprises whey protein.

19. The composition of claim 15, wherein said at least one skin exfoliation ingredient further comprises a polyhydroxy acid.

20. The composition of claim 19, wherein said polyhydroxy acid comprises gluconolactone.

21. The composition of claim 15, wherein said at least one moisturizing ingredient is chosen from hyaluronic acid, astrocaryum murumuru butter, and combinations thereof.

22. The composition of claim 15, wherein said at least one anti-inflammatory ingredient comprises resveratrol.

23. The composition of claim 15, wherein said composition is encapsulated in a lecithin containing liposome.

24. The composition of claim 15, further comprising at least one ingredient that provides protection UVA/UVB protection to achieve an SPF factor of at least 15.

25. The composition of claim 24, wherein said at least one ingredient that provides protection UVA/UVB protection comprises one or more of the following components:
Homosalate;
Ethylhexyl Methoxycinnamate;
Ethylhexyl Salicylate;
Butyl Methoxydibenzoylmethane; and
Octocrylene.

26. The composition of claim 15, further comprising at least one fine line and wrinkle reducer.

27. The composition of claim 15, wherein said at least one fine line and wrinkle reducer comprises a peptide chosen from Palmitoyl Oligopeptide, Palmitoyl Tetrapeptide-7, and combinations thereof.

28. The composition of claim 15, wherein said composition is in the form of a cream, lotion, mousse, serum, or gel.

29. The composition of claim 15, which is a skin cleanser and further comprises a surfactant.

30. The composition of claim 29, wherein said surfactant is sulfate free and comprises coco glucoside, cocoyl sarcosine, or combinations thereof.

31. A method of treating the skin, said method comprising applying to the skin the composition of claim 1.

32. A method of treating the skin, said method comprising applying to the skin the composition of claim 15.
